(19) United States
(12) Patent Application Publication
(10) Pub. No.: US 2024/0277923 A1
(43) Pub. Date: Aug. 22, 2024

(54) Transcutaneous Drug Delivery System with Pump Backpack

(71) Applicant: Zyno Medical, LLC, Natick, MA (US)

(72) Inventors: Chao Young Lee, Weston, MA (US); Zhenhua Mao, Andover, MA (US); James Zhimin Yan, Dedham, MA (US); Mei Zhang, Sharon, MA (US)

(21) Appl. No.: 18/443,649

(22) Filed: Feb. 16, 2024

Related U.S. Application Data
(60) Provisional application No. 63/446,969, filed on Feb. 20, 2023.

(51) Int. Cl. A61M 5/142; A61M 5/14; A61M 5/158

(52) U.S. Cl. CPC A61M 5/14248; A61M 5/1413; A61M 5/158; A61M 2005/14252; A61M 2005/1586

(57) ABSTRACT

An ambulatory pump having a disposable drug container comprised of a flexible IV-type bag sealed to a length of tubing extending from the IV bag. The IV bag is contained in a lightweight rigid housing facilitating attachment to the skin and providing a guide channel for the IV tube against a retaining wall. This allows a motorized backpack to be simply installed on the rigid housing of the drug container for peristaltic pumping of the medicine through the IV tube during use, and then removed from the drug container for reuse with a different drug container, minimizing the overall system cost.

### CROSS REFERENCE TO RELATED APPLICATION

This application claims the benefit of U.S. Provisional Patent Application No. 63/446,969, filed Feb. 20, 2023, which is incorporated by reference herein in its entirety.

### BACKGROUND OF THE INVENTION

The present invention relates to devices for the automatic delivery of drugs to a patient and, in particular, to a drug delivery device allowing field loading of drugs.

The ability to meter intravenous drugs over a long period of time can provide better efficacy of the drug and less toxicity to a patient. For this purpose, ambulatory infusion pumps have been developed, for example, that can be carried on the patient in a harness or the like together with a reservoir of medicament, for example, in an IV-type of bag. An example of such system is described in U.S. Pat. No. 10,507,283, entitled “Medical Pump System for Improved Pain Management”, assigned to the assignee of the present application and hereby incorporated by reference.

An improved class of ambulatory pumps may attach directly to the patient's skin eliminating the need for a harness. Pumps of this kind normally include a pre-charged canister having small quantities of the particular drug and thus present shelf-life and inventory challenges as well as limitations with respect to providing different volumes of drugs.

U.S. patent application Ser. No. 17/894,326, filed Aug. 24, 2022, assigned to the assignee of the present application and hereby incorporated by reference, describes an improved design having a canister with a piston-type pump and valving system so that it can be filled in the field prior to use (for example, by a pharmacist) thereby addressing the shelf-life problem of pre-charged canisters.

### SUMMARY OF THE INVENTION

The present invention provides an ambulatory pump having a disposable drug container comprised of a flexible IV-type bag sealed to a length of tubing extending from the IV bag. The IV bag and tubing provide a robust leak resistant volume that is easily filled by a pharmacist through the IV tube. The IV bag is contained in a lightweight rigid housing facilitating attachment to the skin and providing a guide channel for the IV tube against a retaining wall. This allows a motorized backpack to be simply installed on the rigid housing of the drug container for peristaltic pumping of the medicine through the IV tube during use, and then removed from the drug container for reuse with a different drug container, minimizing the overall system cost.

In one embodiment, the present invention provides a transcutaneous drug delivery system receiving an intravenous (IV) bag containing a liquid comprising a pump housing attachable to skin of a patient and supporting a peristaltic pump providing actuating finger elements; an IV line coupled to the IV bag and communicating with the peristaltic finger elements to provide undulating movement along the IV line; and an IV line connector providing a leak-free connection between the IV line and the IV line connector and coupled to a hypothermic needle to fluidly communicate the liquid to the patient through the hypothermic needle.

It is thus a feature of one embodiment of the present invention to provide a lightweight, ambulatory infusion pump that can be easily worn on the patient's skin during drug delivery so that the patient can conduct normal activities during drug delivery.

The housing may include a first compartment receiving the IV bag and a second compartment supporting the ambulatory pump.

It is thus a feature of one embodiment of the present invention to allow the pump housing and used IV bag to be disposed while the ambulatory pump and electronics can be reused with a replacement pump housing preloaded with a replacement IV bag.

The IV line may pass from the first compartment to the second compartment. The first compartment and second compartment may be separated by a false bottom.

It is thus a feature of one embodiment of the present invention to separate the electronics from the IV bag using a leak-proof barrier to avoid damage to the electronics if there is a leak.

The housing may include a guide channel holding the IV line against a retaining wall.

The peristaltic finger elements may be positioned to undulate towards the retaining wall. The pump may comprise a motor configured to rotate a pump crank.

It is thus a feature of one embodiment of the present invention to secure the IV line between the retaining wall and the peristaltic finger elements to ensure full compression of the IV line regardless of the size of the IV line.

An adhesive sheet may be attached to the pump housing permitting the attachment of the pump housing to the skin of the patient. The adhesive sheet may be oxygen permeable.

It is thus a feature of one embodiment of the present invention to allow the pump housing to be secured to the patient's skin for an extended period but remain easily removable.

The peristaltic pump may be supported on a support board and may further comprise retention elements permitting the support board to be removably released from the pump housing. The retention elements may comprise a latch and tab interlockable to releasably secure the support board to the pump housing.

It is thus a feature of one embodiment of the present invention to allow the peristaltic pump and electronics to be easily removed from the used pump housing and reused within a new pump housing.

The IV line connector may be a Luer-Lock connector providing a plug.

It is thus a feature of one embodiment of the present invention to allow drugs to be loaded into the IV bag through the IV line connector when the plug is removed and for drugs to be expelled through the IV line connector when the plug is removed.

The pump housing may be rigid. The pump housing may be manufactured of a thermoplastic material.

It is thus a feature of one embodiment of the present invention to permit the pumping elements and IV line to provide a hard outer shell protecting the peristaltic elements during movement and prevent the IV line from kinking.

The pump housing may be one inch by one and one half inches in size or smaller.

It is thus a feature of one embodiment of the present invention to allow the pump housing to be comfortably worn on the patient's skin for extended periods of time.

The IV line is at least partially manufactured of a silicone material.

It is thus a feature of one embodiment of the present invention to facilitate movement of the liquid through the IV line by providing a more flexible and soft material at the contact area of the peristaltic fingers.

A method of transcutaneously delivering a drug from an IV bag containing a liquid comprising attaching a pump housing providing an ambulatory pump to skin of a patient, the peristaltic pump having finger elements providing undulating movement of the liquid through the IV line, and an IV line connector providing a leak-free connection between the IV line and the IV line connector and coupled to a hypothermic needle to fluidly communicate the liquid to the patient through the hypothermic needle; connecting the IV line connector to the hypothermic needle; operating the peristaltic pump to undulate the peristaltic finger elements to provide movement of the liquid through the IV line; and expelling the liquid from the IV bag through the IV line and IV line connector into the skin of the patient.

The method may further include filling the IV bag with the liquid drug through the IV line and IV line connector.

The method may further include controlling a flow rate of the liquid from the IV bag.

The peristaltic pump may be supported on a support board. The method may further include removing the support board and peristaltic pump from the pump housing.

These particular objects and advantages may apply to only some embodiments falling within the claims and thus do not define the scope of the invention.

### BRIEF DESCRIPTION OF THE DRAWINGS

FIG. 1 is a fragmentary, perspective view of the ambulatory pump of the present invention as attached to the skin of a patient to communicate with a drug port or the like;

FIG. 2 is an exploded, perspective view of the skin side of a drug container portion of the ambulatory pump of FIG. 1 showing a housing for containing a flexible IV bag and integrated IV line and an adhesive panel for attachment of the drug container to the patient's skin;

FIG. 3 is a front, elevational view of the ambulatory pump of FIGS. 1 and 2 with a cover removed showing a pump backpack that can be removably installed on the drug container portion for peristaltic pumping of the drug;

FIG. 4 is a perspective, top view of the pump of FIGS. 1-3 showing the pump backpack during removal to reduce the marginal costs of the delivery system; and

FIG. 5 is a perspective, top view of the pump of FIGS. 1-3 showing the pump backpack and cover attached to the drug container using a latch.

### DETAILED DESCRIPTION OF THE PREFERRED EMBODIMENT

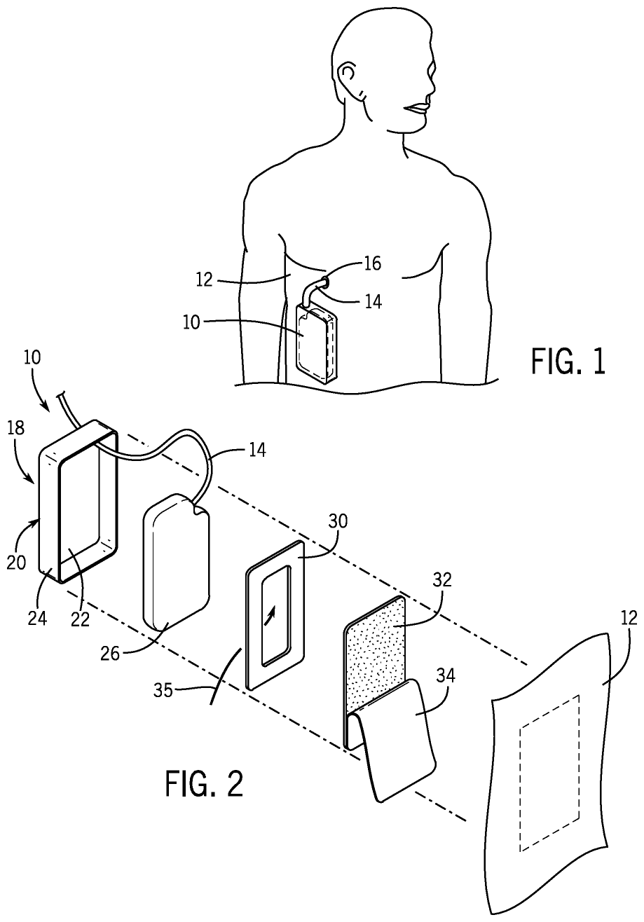

Referring now to FIG. 1, an ambulatory pump 10 according to the present invention may attach, for example, to the skin of a patient 12 to support the ambulatory pump 10 during normal activities of the patient and to provide for the delivery of drugs through an IV line 14 and a drug delivery port 16 or another similar element attached to the patient's skin.

The ambulatory pump 10 may take a small form factor, for example, being approximately one inch by one and one half inches in size or smaller so that it can be carried by the patient's skin.

Referring to FIG. 2, the ambulatory pump 10 may include a low-cost drug container 18 intended for disposal after use with a single patient. The drug container 18 includes a rigid housing frame 20 having a false bottom 22 surrounded by rearwardly and frontwardly extending sidewalls 24. The rearward extent of the sidewalls 24 define a volume into which a flexible IV bag 26 may fit. The IV bag 26 may have an integrally attached IV tube 14 that may extend from the IV bag 26 through an opening in the false bottom 22 from a rear side of the false bottom 22 to a front side of the drug container 18 as will be discussed below.

A retainer ring 30 may fit over the IV bag 26 on the rear side of the false bottom 22, the retainer ring 30 attached at its periphery to the walls 24, to hold the IV bag 26 within a volume defined by the false bottom 22, walls 24, and retainer ring 30. At the time of manufacture, the IV bag 26 may be sterilized and evacuated to be later filled by a pharmacist as will be discussed. The retainer ring 30 may be attached permanently to the retaining walls 24 by any number of well-known manufacturing techniques.

In one embodiment, the drug container 18 including the walls 24, the false bottom 22, and the retainer ring 30 will be a rigid polymer material such as a thermoplastic fabricated by injection molding. The retainer ring 30 may be, for example, a polycarbonate film. The IV bag 26 may be, for example, a flexible vinyl material.

Referring still to FIG. 2, a double-sided adhesive sheet 32 may be attached to the rear surface of the retainer ring 30 by adhesive on its first side. The adhesive sheet 32 may extend to the periphery of the retainer ring 30 and cover a central opening of the retainer ring 30 to provide a broad area of attachment to the patient's skin. The adhesive of the patient-side of the double-sided adhesive sheet 32 may be protected by a siliconized release liner 34 to be removed by the patient prior to adhering the drug container 18 to his or her skin.

The adhesive sheet 32 will desirably be oxygen permeable to allow long-term attachment to the patient 12 while preserving skin health and for this purpose, the center of the retainer ring 30 may allow for the infiltration of outside air 35 to improve oxygen transmission through the adhesive sheet 32, this outside air passing through channels in the retainer ring 30 and/or through openings in the false bottom 22.

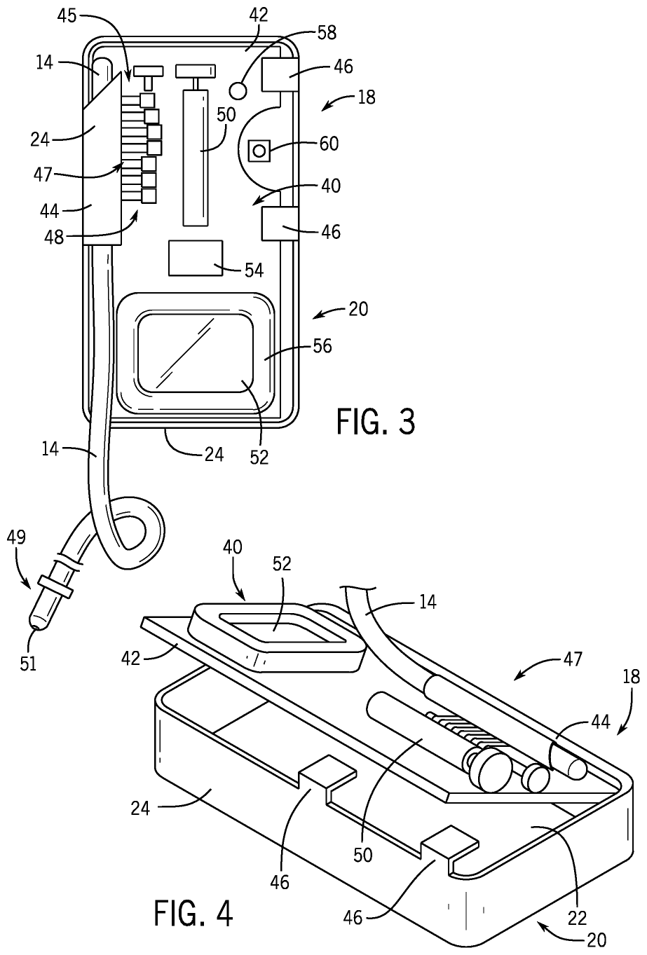

Referring now to FIGS. 3 and 4, a pump backpack 40 may be constructed on a support board 42 that can be snapped into a front of the drug container 18 against the false bottom 22 for pumping of medicament from the IV bag 26 through the IV tube 14. The IV tube 14, after passing through the false bottom 22 may pass along an inner surface of the sidewall 24, for example, held under a ledge 44 on one side of the drug container 18. This same sidewall 24 may provide inwardly extending retainer elements (not shown) holding the support board 42 and allowing the support board 42 to be pivoted downward and then releasably captured under retention tabs 46 on the other side of the drug container 18. In one embodiment, these retention tabs 46 that can be flexibly displaced to allow later removal of the pump backpack 40 after use by the patient 12 and disposal of the drug container 18. For example, a finger actuated button or switch may help with releasing the pump backpack 40 from the retention tabs 46. In one embodiment, as described below, the retention tabs 46 may interact with a flexible latch 66 of the pump backpack 40 to allow for removal of the pump backpack 40 from the drug container 18.

When the pump backpack 40 is installed on the drug container 18, the IV tubing 14 passing along the sidewall 24 under the ledge 44 is sandwiched between the inner surface of this sidewall 24 and multiple undulating fingers 45 of a peristaltic pump 47, these fingers 45 driven by a pump crank 48 having associated cams. The fingers 45 may extend at least partially underneath the ledge 44 in order to undulate toward the IV tubing 14 in a reciprocating manner.

The IV tube 14 then proceeds downward as depicted and out through an opening in the sidewalls 24 of the drug container 18 to a connector 49 such as a Luer-Lock having a plug 51. Prior to use by the patient 12, the plug 51 may be removed and the IV bag (shown in FIG. 2) filled by a pharmacist with reverse flow through the IV tube 14 into the IV bag 26. Then the plug 51 may be reinstalled for delivery to the patient 12 who will remove the plug 51 and connect the device to his or her skin and the port 16 or similar device on the patient's skin. Connectors and adapters of the type known in the art may assist with attachment of an end of the IV tube 14 to a drug delivery system which delivers the drug under the patient's skin using a hypodermic needle.

The IV tubing 14 may be a soft silicone material to facilitate this peristaltic pumping or may be a combination of a more rigid vinyl and silicone materials with the silicone materials positioned only in the area of fingers 45.

The pump crank 48 may be turned by a DC electric motor 50 powered by a rechargeable lithium-ion battery 52 or the like under the control of electronics 54 supported by the support board 42 which may provide for printed circuitry. The pump crank 48 may be turned by a series of connected gears and/or shafts of the DC electric motor 50. The rotation of the pump crank 48 may be metered to expel a specific volume of drug with every rotation of the DC electric motor 50 and/or pump crank 48. A motor sensor (not shown) may detect the number of rotations of the gears and/or shafts of the DC electric motor 50 and/or pump crank 48 to calculate an expelled drug volume from the flexible IV bag 26 through the IV tubing 14 and which may in part depend on the size of the IV tubing 14.

A recharging coil 56 may be attached to the battery 52, for example, operating under the Qi wireless charging standard commonly used for cell phones and the like to allow wireless recharging of the battery 52.

The control electronics 54 may provide for a preprogrammed flow rate of medicament after activation by the patient 12, for example, by user accessible switch 58 or may include a Bluetooth or other wireless connection to allow more sophisticated control using the patient's smart phone or other similar device. If there is no Bluetooth or wireless connection, the control electronics 54 may operate to deliver a preprogrammed flow rate until the flexible IV bag 26 is empty or all the drugs have been dispensed. A fluid sensor may indicate when the IV bag 26 is empty or no fluid is flowing through the IV tube 14 indicating a bag empty state.

A top side of the false bottom 22 may include a switch 60, for example, a tactile switch or dome switch that is closed when the support board 42 is installed on the drug container 18 and communicating to the control electronics 54 to implement a lockout of motor operation when such installation is not indicated.

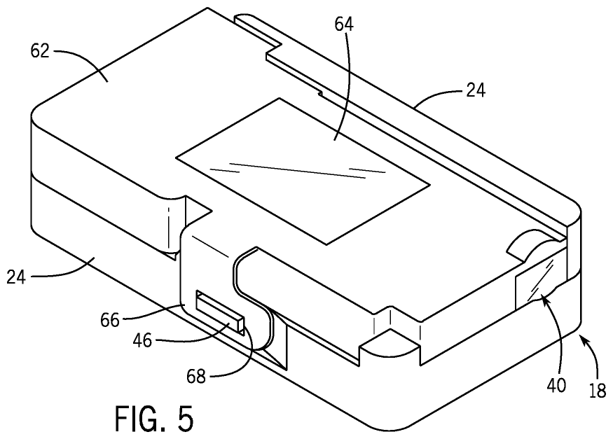

Referring to FIG. 5, in one embodiment, the pump backpack 40 may include a cover 62 or a separate cover may be installed over the pump backpack 40 and the drug container 18 to protect the moving elements of the pump 10 from clothing and the like and to provide a finished appearance. The cover 62 may include a user interface 64 for direct control of the user accessible switch 58 and the control electronics 54. The cover 64 of the pump backpack 40 may be tightly sealed against the top of the support board 42 to protect the electronics 54 from moisture or any accidental fluid leakage for example, emanating from the drug container 18 or IV tubing 14.

The cover 62 may support the support board 42 therein and may be captured underneath the inwardly extending retainer elements of the sidewall 24 to hold the cover 62 on one side while the other side of the cover 62 may include a downwardly extending latch 66 which extends downwardly toward the retention tab 46 of the drug container 18. The retention tab 46 is inserted into an opening 68 of the latch 66 to secure the drug container 18 to the pump backpack 40. The user may manually flex the latch 66 away from the retention tabs 46, or press the retractable retention tab 46, to release the retention tab 46 away from the opening 68 of the latch 66 and thus release the pump backpack 40 from the retention tab 46.

It is understood that the pump backpack 40 can be reused with different drug containers 18 that may be tailored for different types of drug deliveries. The drug container 18 may hold different types of drugs and different amounts of drugs, for example, 1 mL to 100 mL. The drug container 18 can thus vary in size, for example, being smaller or larger than the pump backpack 40 without affecting the connection of the drug container 18 to the pump backpack 40 and without affecting the placement of the IV tubing 14 underneath the area of the fingers 45 for peristaltic pumping.

Various drug delivery protocols may be supported by the control electronics 54 and the present invention contemplates that the ambulatory pump 10 may be comfortably worn by the patient for several days or even weeks depending on the drug delivery protocol. In some embodiments, a single drug bolus may be delivered or drugs may be delivered periodically over an extended time.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A transcutaneous drug delivery system receiving an intravenous (IV) bag containing a liquid comprising:
a pump housing attachable to skin of a patient and supporting
a peristaltic pump providing actuating finger elements;
an IV line coupled to the IV bag and communicating with the peristaltic finger elements to provide undulating movement along the IV line; and
an IV line connector providing a leak-free connection between the IV line and the IV line connector and coupled to a hypothermic needle to fluidly communicate the liquid to the patient through the hypothermic needle.

2. The transcutaneous drug delivery system of claim 1 wherein the housing includes a first compartment receiving the IV bag and a second compartment releasably supporting the peristaltic pump.

3. The transcutaneous drug delivery system of claim 2 wherein the IV line passes from the first compartment to the second compartment.

4. The transcutaneous drug delivery system of claim 3 wherein the first compartment and second compartment are separated by a false bottom.

5. The transcutaneous drug delivery system of claim 1 wherein the housing includes a guide channel holding the IV line against a retaining wall.

6. The transcutaneous drug delivery system of claim 5 wherein the peristaltic finger elements are positioned to undulate towards the retaining wall.

7. The transcutaneous drug delivery system of claim 6 wherein the peristaltic pump comprises a motor configured to rotate a pump crank.

8. The transcutaneous drug delivery system of claim 1 further comprising an adhesive sheet attached to the pump housing permitting attachment of the pump housing to the skin of the patient.

9. The transcutaneous drug delivery system of claim 8 wherein the adhesive sheet is oxygen permeable.

10. The transcutaneous drug delivery system of claim 1 wherein the peristaltic pump is supported on a support board and the pump housing further comprises retention elements permitting the support board to be removably released from the pump housing.

11. The transcutaneous drug delivery system of claim 10 wherein the retention elements comprise a latch and tab interlockable to releasably secure the support board to the pump housing.

12. The transcutaneous drug delivery system of claim 1 wherein the IV line connector is a Luer-Lock connector providing a plug.

13. The transcutaneous drug delivery system of claim 1 wherein the pump housing is rigid.

14. The transcutaneous drug delivery system of claim 13 wherein the pump housing is manufactured of a thermoplastic material.

15. The transcutaneous drug delivery system of claim 1 wherein the pump housing is one inch by one and one half inches in size or smaller.

16. The transcutaneous drug delivery system of claim 1 wherein the IV line is at least partially manufactured of a silicone material.

17. A method of transcutaneously delivering a liquid drug from an IV bag containing the liquid drug comprising:
attaching a pump housing to skin of a patient, the pump housing supporting a peristaltic pump, the peristaltic pump having finger elements providing undulating movement of the liquid through the IV line, and an IV line connector providing a leak-free connection between the IV line and the IV line connector and couplable to a hypothermic needle to fluidly communicate the liquid drug to the patient;
connecting the IV line connector to the hypothermic needle;
operating the peristaltic pump to undulate the peristaltic finger elements to provide movement of the liquid through the IV line; and
expelling the liquid from the IV bag through the IV line and IV line connector into the skin of the patient.

18. The method of claim 17 further comprising filling the IV bag with the liquid drug through the IV line prior to attaching the pump housing to the skin of the patient.

19. The method of claim 17 further comprising controlling a flow rate of the liquid from the IV bag.

20. The method of claim 17 wherein the peristaltic pump is supported on a support board and further comprising removing the support board and peristaltic pump from the pump housing following drug delivery.
